(19) United States
(12) Patent Application Publication
(10) Pub. No.: US 2014/0356970 A1
(43) Pub. Date: Dec. 4, 2014

(54) Liquid crystal devices with mixed anion metal salts

(71) Applicant: Wisconsin Alumni Research Foundation, Madison, WI (US)

(72) Inventors: Nicholas Lawrence Abbott, Madison, WI (US); Jacob Tyler Hunter, Madison, WI (US)

(21) Appl. No.: 13/906,779

(22) Filed: May 31, 2013

(51) Int. Cl. G01N 21/84; G01N 33/20

(52) U.S. Cl. CPC G01N 21/84; G01N 33/20; G01N 2021/8477
USPC 436/104; 422/83

(57) ABSTRACT

Methods and devices for determining a preferred composition of anions in a liquid crystal device, and methods and devices for detecting a targeted agent in a sample using such preferred compositions, are disclosed. Such methods include the steps of preparing a liquid crystal device including a liquid crystal, a metal cation, a strongly coordinating anion, and a weakly coordinating anion, contacting the liquid crystal device with a sample suspected of including the targeted agent, and observing the orientational ordering of the liquid crystal at the surface.

### STATEMENT REGARDING FEDERALLY SPONSORED RESEARCH OR DEVELOPMENT

This invention was made with government support under 1121288 awarded by the National Science Foundation and W911NF-11-1-0251 and W911NF-10-1-0181 awarded by the ARMY/ARO. The government has certain rights in the invention.

### CROSS REFERENCE TO RELATED APPLICATIONS

N/A

### FIELD OF THE INVENTION

The disclosure relates generally to methods and devices for determining a preferred composition of anions in a liquid crystal device, and methods and devices for detecting a targeted agent in a sample. The invention further relates to the use of mixtures of metal salts with varying proportions of anions to improve the sensitivity and selectivity of liquid crystal devices for the detection of a targeted agent.

### BACKGROUND OF THE INVENTION

Liquid crystal (LC)-based sensor devices for detecting targeted agents are well-known in the art. Such devices may include thin films of nematic LCs supported on a chemically functionalized surface. If the surface is functionalized with a metal salt, interactions between the metal salts and LCs result in long-range alignment of the LC molecules, which is readily observed under a microscope with a polarized light source or by using other methods known in the art. Observed changes in the alignment of the LC molecules may signal the presence of a targeted agent. For example, U.S. Pat. Pub. No. 2007/0004046, which is incorporated by reference herein in its entirety, discloses that dimethylmethylphosphonate, or DMMP, induces a change from a homeotropic to a planar alignment in the orientation of 4-pentyl-4′-cyanobiphenyl (5CB) films or other nitrile containing LCs such as E7 in contact with aluminum (III) perchlorate salts decorated on solid surfaces.

A wide range of targeted compounds can be detected using LC-based sensors, including solids, liquids and gases. Non-limiting examples of gases that can be detected using such sensors include amine containing compounds, nitrogen oxides, hydrogen disulfide and other sulfur containing compounds (such as half mustard), volatile organic compounds, organophosphates, and organophosphonates. As a non-limiting example, the organophosphates and organophosphonates include a variety of compounds that are of interest as targeted agents for detection. This class of compounds includes nerve toxins, such as sarin, that could potentially be used by terrorists and as a weapon of war, and certain pesticides, such as malathion.

Many organophosphates and organophosphonates are highly toxic, even at low concentration. For example, the sarin concentration that is lethal in 50% of humans when delivered by inhalation is 10-15 ppm min⁻¹. Thus, its is vital that methods of detecting such agents have very high sensitivity and selectivity. Previously disclosed LC-based methods using 100% aluminum perchlorate metal salt disposed on a surface have a maximum sensitivity for detection of DMMP of about 250 ppb. Furthermore, such methods are subject to the effects of humidity, which can substantially reduce the efficacy of metal salt-based LC sensors. Thus, there is a need in the art for LC-based sensors having greater selectivity and sensitivity for targeted agents.

### BRIEF SUMMARY

As a result of the surprising discovery that mixtures of anions of varying coordination strength can be used to substantially improve both the sensitivity and selectivity of LC-based sensor devices, the inventors have developed LC-based sensor devices that include compositions of mixed metal salts having a combination of weakly coordinating and strongly coordinating anions, as well as associated methods of detecting a targeted agent and methods of optimizing the composition of mixed metal salts. The disclosed methods and devices can be used to detect a wide variety of target compounds, and are not limited in terms of the class of compound that can be detected.

Accordingly, in a first aspect, the disclosure encompasses a device for detecting a targeted agent. The device includes a substrate, one or more mixed metal salts disposed on the substrate, and a liquid crystal in contact with the mixed metal salts. The mixed metal salts each include one type of metal cation and two or more types of anions. In some embodiments, the two or more types of anions include at least one strongly coordinating anion and at least one weakly coordinating anion.

Non-limiting examples of strongly coordinating anions that could be used in the device include CH₃COO⁻, C₅H₇O₂⁻ (acetylacetonate), Cl⁻, F⁻, Br⁻, SO₄²⁻, and NO₃⁻. Non-limiting examples of weakly coordinating anions that could be used in the device include ClO₄⁻, BF₄⁻, PF₆⁻, SbF₆⁻, and B(OTeF₅)⁻. In some embodiments, the weakly coordinating anion is ClO₄⁻. In some embodiments, the strongly coordinating anion is NO₃⁻ or Cl⁻ and the weakly coordinating anion is ClO₄⁻. In some embodiments, the strongly coordinating anion is acetylacetonate and the weakly coordinating anion is ClO₄⁻.

Non-limiting examples of metal cations that could be used in the device include Al³⁺, Ag¹⁺, Ba³⁺, Cd²⁺, Ce³⁺, Co²⁺, Cr³⁺, Cu²⁺, Eu³⁺, Fe²⁺, Fe³⁺, Ga³⁺, In³⁺, La³⁺, Mn²⁺, Ni²⁺, Pb²⁺, Pr³⁺, and Zn²⁺. In some embodiments, the metal cation is Al³⁺ or Cu²⁺. In some embodiments, the metal cation is Al³⁺.

In some embodiments, the strongly coordinating anion makes up from about 5% to about 95% of the total moles of weakly coordinating anions and strongly coordinating anions present in the mixed metal salt. In some embodiments, the strongly coordinating anion makes up from about 50% to about 92% of the total moles of weakly coordinating anions and strongly coordinating anions present in the mixed metal salt. In some embodiments, the strongly coordinating anion makes up from about 70% to about 91% of the total moles of weakly coordinating anions and strongly coordinating anions present in the mixed metal salt. In some embodiments, the strongly coordinating anion makes up from about 75% to about 90% of the total moles of weakly coordinating anions and strongly coordinating anions present in the mixed metal salt. In some embodiments, the strongly coordinating anion makes up about 90% of the total moles of weakly coordinating anions and strongly coordinating anions present in the mixed metal salt. In some embodiments, the strongly coordinating anion makes up about 75% of the total moles of weakly coordinating anions and strongly coordinating anions present in the mixed metal salt.

In some embodiments, the metal cation is immobilized on the substrate. In a non-limiting example of such an embodiment, the metal cation is immobilized on the substrate by binding to a carboxylic acid moiety that is linked to the substrate. Optionally, the carboxylic acid moiety can be linked to the substrate through a self-assembled monolayer (SAM) comprising a carboxylic acid moiety. In some embodiments, the substrate may include a solid surface.

Examples of specific liquid crystals that can be used in the device include without limitation 5CB (4-n-pentyl-4′-cyanobiphenyl) or 8CB (4-cyano-4′octylbiphenyl).

In some embodiments, the device further includes a means for observing the orientational ordering of the liquid crystal.

In a second aspect, the disclosure encompasses a method for detecting a targeted agent in a sample. The first step of the method is preparing a liquid crystal device that includes a substrate, one or more mixed metal salts disposed on the substrate, and a liquid crystal in contact with the mixed metal salts, as described above. Optionally, the device may include one or more or the additional features described above. The method further includes the steps of contacting the liquid crystal in the device with the sample, and observing the orientational ordering of the liquid crystal in the device. A change in the orientational ordering of the liquid crystal in the device indicates that the targeted agent is present in the sample.

In some embodiments, the change in orientation ordering of the liquid crystal indicating the presence of the targeted agent is a change from homeotropic alignment to planar alignment. In some embodiments, the sample may be in the gas phase.

Optionally, the targeted agent may be an organophosphate or an organophosphonate. Non-limiting examples of organophosphates that could be used as targeted agents include (RS)-Propan-2-yl methylphosphonofluoridate (sarin), Cyclohexyl methylphoshonofluoridate (cyclosarin), 3,3-Dimethylbutan-2-ylmethylphosphonofluoridate (soman), (RS)-Ethyl N,N Dimethylphosphoramidocyanidate (tabun), dimethyl methylphosphonate (DMMP), O-ethyl S-[2-(diisopropylamino)ethyl]methylphosphonothioate (VX), Diethyl 2-[(dimethoxyphosphorothioyl)sulfanyl]butanedioate (malathion), O,O-Diethyl O-[4-methyl-6-(propan-2-yl)pyrimidin-2-yl]phosphorothioate (diazinon), O,O-Diethyl O-(4-nitrophenyl) phosphorothioate (parathion), and O,O-Dimethyl O-(4-nitrophenyl) phosphorothioate (methyl parathion).

In a third aspect, the disclosure encompasses a method for determining an optimal mole fraction of strongly coordinating anion in a mixed metal salt for use in a liquid crystal device for detecting a targeted agent. The method includes the steps of (a) preparing two or more compositions comprising a mixed metal salt comprising one type of metal cation, a strongly coordinating anion, and a weakly coordinating anion, wherein each of the two more or more compositions includes a different proportion of strongly coordinating anion and weakly coordinating anion, as calculated by the mole fraction of the total moles of weakly coordinating anion and strongly coordinating anion present; (b) disposing the two or more compositions onto separate substrates or onto separate regions of the same substrate; (c) contacting the two or more compositions with a liquid crystal; and (d) observing the orientational ordering of the liquid crystal in contact with each of the two or more compositions.

The optimal mole fraction of the strongly coordinating anion is the mole fraction of strongly coordinating anion in the composition having the highest mole fraction of strongly coordinating anion that still exhibits a desired initial liquid crystal orientation. In some embodiments, the desired initial liquid crystal orientation is homeotropic liquid crystal alignment.

Non-limiting examples of strongly coordinating anions that could be used in the method include CH₃COO⁻, C₅H₇O₂⁻ (acetylacetonate), Cl⁻, F⁻, Br⁻, SO₄²⁻, and NO₃⁻. Non-limiting examples of weakly coordinating anions that could be used in the method include ClO₄⁻, BF₄⁻, PF₆⁻, SbF₆⁻, and B(OTeF₅)⁻. In some embodiments, the weakly coordinating anion is ClO₄⁻. In some embodiments, the strongly coordinating anion is NO₃⁻ or Cl⁻ and the weakly coordinating anion is ClO₄⁻. In some embodiments, the strongly coordinating anion is acetylacetonate and the weakly coordinating anion is ClO₄⁻.

Non-limiting examples of metal cations that could be used in the method include Al³⁺, Ag¹⁺, Ba³⁺, Cd²⁺, Ce³⁺, Co²⁺, Cr³⁺, Cu²⁺, Eu³⁺, Fe²⁺, Fe³⁺, Ga³⁺, In³⁺, La³⁺, Mn²⁺, Ni²⁺, Pb²⁺, Pr³⁺, and Zn²⁺. In some embodiments, the metal cation is Al³⁺ or Cu²⁺. In some embodiments, the metal cation is Al³⁺.

In some embodiments, the metal cation is immobilized on the substrate. In a non-limiting example of such an embodiment, the metal cation is immobilized on the substrate by binding to a carboxylic acid moiety that is linked to the substrate. Optionally, the carboxylic acid moiety can be linked to the substrate through a self-assembled monolayer (SAM) comprising a carboxylic acid moiety. In some embodiments, the substrate may include a solid surface.

Further objects, features and advantages of the invention will be apparent from the following detailed description when taken in conjunction with the accompanying drawings.

### BRIEF DESCRIPTION OF THE DRAWINGS

The disclosure will be better understood and features, aspects and advantages other than those set forth above will become apparent when consideration is given to the following detailed description thereof. Such detailed description makes reference to the following drawings.

FIG. 1. The alignment of thin films of various LC films at various metal salt decorated surfaces taken from various past studies. The solid square data points represent homeotropic alignment of the LC and the open circle data points represent a planar alignment of the LC.

FIG. 2. (A) Measurement of the tilt angle from surface normal of 5CB supported by perchlorate/nitrate mixtures (solid points) and perchlorate/acetylacetonate mixtures (open squares). The critical compositions of the perchlorate/nitrate mixture is the solid black line and the perchlorate/acetylacetonate mixture is the hyphenated line. The optical micrographs of representative samples for 5CB films in contact with Al (III) salts composed of (B) 100% perchlorate salt, (C) a 8% nitrate/92% perchlorate salt mixture and (D) 100% nitrate salt. The scale bars represent 1 mm. (E) The calculated tilt angle of 5CB at solid surfaces decorated with mixtures of copper (II) perchlorate and copper (II) chloride for sandwich cells of 18 um thick films of 5CB.

FIG. 3. PM-IRRAS spectra of 8CB films supported on copper (II) salts of mixed anion composition. PM-IRRAS spectra are shown for 100% perchlorate sample (solid line), a 50% perchlorate, 50% chloride sample (dashed line), and a 100% chloride sample (dotted line).

FIG. 4. Polarized light images for three independent LC sensor systems with different mixtures of the nitrate and perchlorate anions exposed to 1 ppm DMMP at 0 seconds. The number above each column indicates the time (seconds) of the polarized micrograph. (B) The normalized light intensities for the images shown in B indicating the change in the light intensity occurs after 1 ppm DMMP flowing at 1000 mL/min is introduced into the system at 0 sec. The data points represent a 10%/90% perchlorate/nitrate aluminum (III) salt (solid squares), a 50%/50% perchlorate/nitrate aluminum (III) salt (solid circles), and a 100% perchlorate aluminum (III) salt (solid triangles).

FIG. 5. (A) The time of response, tₒₙ, for perchlorate nitrate mixtures as the concentration of DMMP in the vapor phase varies. Data is plotted for 100% perchlorate salt surfaces (diamonds), 50%/50% nitrate/perchlorate salt surfaces (squares), and 90%/10% nitrate/perchlorate salt surfaces (triangles). (B) The time of response for perchlorate and acetylacetonate are shown. Data is plotted for a 100% perchlorate salt surface (diamonds) and a 50%/50% perchlorate/acetylacetonate salt surface (squares).

FIG. 6. The calculated sensitivities (Cₘᵢₙ) for various salt surfaces that were calculated using the model described from Equation 1 and the data shown in FIG. 4.

FIG. 7. The time of response for a 100% perchlorate and a 50% perchlorate/50% nitrate salt surfaces (mole percent) under 30% humidity (white bars) and 60% humidity (black bars) conditions at a concentration of 500 ppb DMMP.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

### I. In General

Before the present materials and methods are described, it is understood that this invention is not limited to the particular methodology, protocols, materials, and reagents described, as these may vary. It is also to be understood that the terminology used in this disclosure is for the purpose of describing particular embodiments only, and is not intended to limit the scope of the present invention, which will be limited only by the language of the appended claims.

As used in this disclosure and in the appended claims, the singular forms “a”, “an”, and “the” include plural reference unless the context clearly dictates otherwise. The terms “a” (or “an”), “one or more” and “at least one” can be used interchangeably. The terms “comprising”, “including”, and “having” can also be used interchangeably.

Unless defined otherwise, all technical and scientific terms used in this disclosure have the same meanings as commonly understood by one of ordinary skill in the art to which this disclosure belongs. All publications and patents specifically mentioned in this disclosure are incorporated by reference for all purposes, including for describing and disclosing the chemicals, instruments, statistical analysis and methodologies which are reported in the publications which might be used in connection with the disclosed methods and devices. All references cited in this disclosure are to be taken as indicative of the level of skill in the art.

### II. The Invention

Nematic liquid crystals are materials with mobilities characteristic of liquids, but that are capable of organizing over distances of hundreds of micrometers. Past theoretical and experimental studies have established that the orientations of liquid crystals near an interface to a confining medium are dictated by the chemical and topographical structure of that interface. This so-called anchoring of liquid crystals by surfaces has found widespread use in the display industry and underlies the principles that are being developed for the detection of molecular and biomolecular events at interfaces. Specifically, a change in the chemical or topographical structure of an interface brought about by a chemical or biological species at a surface can give rise to new orientations of liquid crystals in contact with that surface. As liquid crystals are birefringent, these new orientations can be visualized under simple polarized microscopy.

This disclosure is based on the inventors' surprising discovery that the use of mixed anions impacts the subsequent interactions of the cation of the metal salt with other species in the system, including the liquid crystal and the targeted agent. That the coordination strengths of the anions in a mixture impacts the ease by which the targeted agent interacts with the metal salt to disturb the coordination complex was not known previously, and could not have been predicted based on what was known in the art. While past studies have shown that the choice of a single anion can change the orientation of a LC on a surface with metal cations, there was no reason to expect, based on this prior observation, that mixtures of two or more anions can be used to achieve anchored LCs that are more sensitive to a targeted agent than a surface covered with the metal salt with a single type of anion. The inventors have unexpectedly observed that the selectivity of the LC system (i.e., the tendency for the LC to be perturbed by one targeted analyte in the presence of another) can be influenced positively by the use of a selected mixture of anions.

The term “liquid crystal,” as used in this disclosure, refers to an organic composition in an intermediate or mesomorphic state between solid and liquid. Suitable liquid crystals for use in the present invention include, but are not limited to, thermotropic liquid crystals. The disclosed methods and devices may employ polymeric liquid crystals, composite materials comprising particles and liquid crystals, or polymers and liquid crystals, as well as elastomeric liquid crystals. The disclosed methods and devices may also use liquid crystalline gels, including colloid-in-liquid crystal gels and molecular liquid crystalline gels containing, for example, gelators comprised of derivatives of amino acids. In certain embodiments of the disclosed methods and devices, the liquid crystal phase can include a low molecular weight liquid crystal, a liquid crystal elastomer, a liquid crystalline gel, or a liquid crystal droplet. The liquid crystal may also contain a chiral additive to create a cholesteric phase.

An example of a liquid crystalline elastomer is synthesized from the mesogen M₄OCH₃ and polymethylhydrosiloxane, according to A. Komp and coworkers “A versatile preparation route for thin free standing liquid single crystal elastomers” Macromol. Rapid Commun, 26: 813-818, 2005. Other LC elastomers suitable for use in the current disclosure are described by Deng in “Advances in liquid crystal elastomers” (Progress in Chemistry, 18 (10): 1352-1360, 2006), and in the documents cited by Deng. The scope of this disclosure also includes the use of liquid crystalline hydrogels, as described by Weiss, F. and Finkelmann H. in Macromolecules; 37(17); 6587-6595, 2004, and in the documents cited by Weiss and Finkelmann. Other embodiments use a composite comprising a dispersion of solid particulates, such as but not limited to microspheres, mixed with liquid crystal. Such composites are known by those skilled in the art to form a gel.

Other classes of liquid crystals that may be used in accordance with the disclosed devices and methods include, but are not limited to: polymeric liquid crystals, thermotropic liquid crystals, lyotropic liquid crystals, columnar liquid crystals, nematic discotic liquid crystals, calamitic nematic liquid crystals, ferroelectric liquid crystals, discoid liquid crystals, liquid crystal mixtures, bent-core liquid crystals, liquid crystals that are achiral to which a chiral dopant was added, and cholesteric liquid crystals. Examples of just some of the liquid crystals that may be used are shown in Table 1. In some embodiments, the liquid crystal is a nematic liquid crystal such as 4-pentyl-4′-cyanobiphenyl

### (5CB):

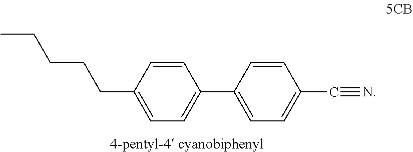 [SMILES: CCCCCc1ccc(-c2ccc(C#N)cc2)cc1]

TABLE 1
Molecular Structure of Mesogens Suitable for Use in the Disclosed Methods and Devices.

| Mesogen | Structure |
|---|---|
| Anisaldazine | 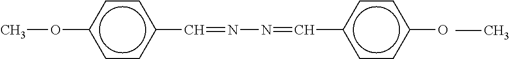 |
| NCB | 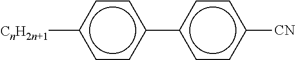 |
| CBOOA | 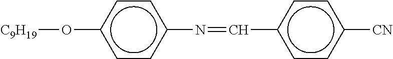 |
| Comp A | 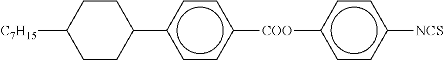 |
| Comp B | 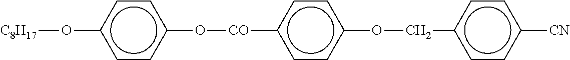 |
| DB₇NO₂ | 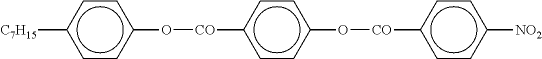 |
| DOBAMBC | 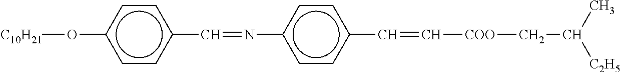 |
| nOm n = 1, m = 4: MBBA n = 2, m = 4: EBBA | 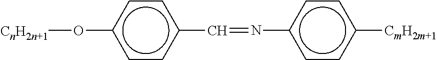 |
| nOBA n = 8: OOBA n = 9: NOBA | 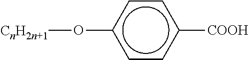 |
| nmOBC | 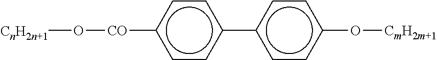 |
| nOCB | 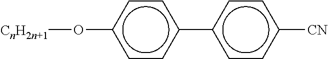 |
| nOSI | 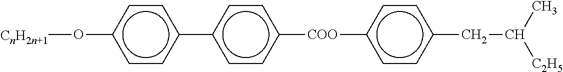 |
| 98P | 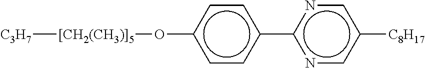 |
| PAA | 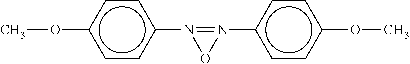 |
| PYP906 | 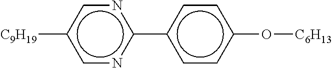 |
| n̅Sm | 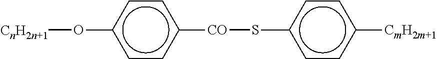 |

In this disclosure, we classify anions as either strongly or weakly coordinating. Strongly coordinating anions are anions that are less stable by themselves and thus have a higher tendency to donate their electrons to an electron acceptor. Such anions are generally smaller species, such as Cl⁻ or NO₃⁻, which possess non-uniform charge distributions. Weakly coordinating anions (sometimes referred to as non-coordinating anions) are much more stable on their own and do not have a propensity to donate the electrons to an electron acceptor. These anions tend to be bulkier, have a uniform charge distribution, and exhibit resonant structures. Nonlimiting examples of weakly coordinating anions include perchlorate (ClO₄⁻) or tetrafluoroborate (BF₄⁻).

The classification of the anions can be based on the interactions of the anions with the hydrogen ion as the electron acceptor. By analyzing the equilibrium of the anion and the H⁻ anion complex the relative stability of the anion versus the complex of the anion with the H⁻ anion can be assessed. This equilibrium is related to the basicity constant of the anion. A very low value indicates that the anion is stable by itself. Thus, in general, an anion having a basicity constant of less than −7.5 is classified as a weakly coordinating anion, and an anion having a basicity constant of greater than −6.5 is classified as a strongly coordinating anion. However, as is recognized in the art, the basicity constant only provides a rough estimate of the coordinating strength of the anion. In practice, the coordinating properties of anions are dependent on the specific electron acceptor in the system and would take into account the differences in size, polarizability, charge, and available orbitals of the electron acceptor.

As used in this disclosure, the term “substrate” refers to an interface upon which another material can be layered or attached. Optionally, a substrate may be a solid surface, but it may also include without limitation a liquid-liquid interface. Solid supports that may form a substrate include, but are not limited to, glass, metals (including gold), gels, and filter paper, among others. In certain embodiments, the substrate may include a surface that is functionalized to include a moiety that binds to the cation of the mixed metal salt.

A method for determining a preferred mixture of anions in a metal salt comprises preparing of a series of substrates or substrate regions that may comprise a solid surfaces each decorated with a metal cation accompanied by an increasing mole fraction of strongly coordinating anion within a mixture of anions (a mixture of weakly and strongly coordinating anions). Subsequently, the orientation of LC on each of the surfaces is determined by methods known to those skilled in the art, including optical methods that employ transmission of polarized light through the supported LC film. The surface decorated with the highest mole fraction of strongly coordinating anion (or close to the highest mole fraction) that gives a desired initial orientation of the LC is identified as the preferred surface. The composition of anions on this surface is an optimal composition for maximizing the sensitivity of the supported LC film to targeted agents. In some embodiments, the desired initial orientation is homeotropic orientation.

A surface or substrate may be “decorated” with a metal salts by, for example, first applying a self-assembled monolayer (SAM) to the surface or substrate. The SAM may include thiol moieties which may interact with the substrate to anchor the SAM to the substrate. The SAM may also include a carboxylic acid moiety which may interact with the metal cation to localize the metal salt on the substrate. One example of a suitable SAM for practicing the present invention is 11-mercaptoundecanoic acid. However, the use of other suitable SAM's, which are well known in the art, are contemplated.

Metal ions which are suitable for the manufacture of liquid crystal devices are well known in the art. The disclosed methods and devices are not limited to the use of any particular metal ion. Indeed, the use of a variety of metal ions is contemplated, including, but not limited to, those selected from the group consisting of Al³⁺, Ag¹⁺, Ba³⁺, Cd²⁺, Ce³⁺, Co²⁺, Cr³⁺, Eu³⁺, Fe²⁺, Fe³⁺, Ga³⁺, In³⁺, Mn²⁺, Ni²⁺, Pb²⁺, Pr³⁺, Cu²⁺, and Zn²⁺.

As is known to those skilled in the art, changes in the orientational order of the liquid crystal can lead to a change in the optical properties of the liquid crystal. Such changes can be detected and quantified by using optical instrumentation such as, but not limited to, plate readers, cameras, scanners, and photomultiplier tubes. Because the dielectric properties of liquid crystals also change with orientational order, measurements of electrical properties of liquid crystals can also be used to report changes in the orientational order of the liquid crystals. Thus a wide range of optical and electrical methods for observing the change in orientational order of liquid crystals is encompassed by this disclosure.

For example, in certain embodiments, the step of observing the orientational ordering of the liquid crystal at the interface is performed by detecting plane polarized light that is passed through the interface or liquid crystal surface. In some such embodiments, the plane polarized light is passed through the interface between crossed polarizers. Homeotropic ordering can be shown by observing the absence of transmitted light between cross-polarizers, and can be confirmed by an interference pattern consisting of two crossed isogyres under conoscopic examination. Planar ordering results in bright colored appearance when viewed between cross-polarizers.

In some embodiments of the disclosed methods and devices, the orientational ordering of the liquid crystal undergoes change over time, as the targeted agent (such as, for example, DMMP) is introduced into the system. Thus, there is a transitional orientational ordering state between the planar orientation (parallel to the LC interface or surface) and the homeotropic orientation (perpendicular to the LC interface or surface). The transitional ordering is indicated by the so-called “tilt angle,” which is the angle at which the LC is oriented as compared to the surface normal (a vector perpendicular to the surface). A change in orientation of the LC can also involve a change in the azimuthal orientation.

In some embodiments, the step of observing the orientational ordering of the liquid crystal at the interface comprises calculating the tilt angle of the liquid crystal at the interface relative to the interface normal. The change in the tilt angle over time indicates the extent of the change in orientational ordering. Methods of calculating the tilt angle are not limited, and include using the effective birefringence of the liquid crystal under white light illumination.

In such embodiments, the color of the LC under white-light illumination can be matched against a Michel-Levy chart to determine the effective birefringence Δn_{eff} of a LC film of known thickness. For each value of Δn_{eff}, the tilt angle at an aqueous-LC interface (measured relative to the surface normal), θ, can be determined by, for example, solution of the equation:

$$Δ {n}_{eff} \approx \frac{1}{d} {\int }_{0}^{d} ( \frac{{n}_{‖} {n}_{⊥}}{\sqrt{{n}_{⊥}^{2} {sin}^{2} ( \frac{z}{d} θ ) + {n}_{‖}^{2} {cos}^{2} ( \frac{z}{d} θ )}} - {n}_{⊥} ) d z ,$$

where n_{∥} and n_{⊥} are the indices of refraction parallel and perpendicular to the optical axis of the LC, respectively, and d is the thickness of the LC film. Alternatively, a compensator can be used to quantify the optical retardance of the LC or a polscope can also be used.

The following example is offered for illustrative purposes only, and is not intended to limit the scope of the invention in any way. Indeed, various modifications of the invention in addition to those shown and described herein will become apparent to those skilled in the art from the foregoing description and the following example and fall within the scope of the appended claims.

### III. Example

### Adsorbate-Induced Anchoring Transitions of Liquid Crystals on Surfaces Presenting Metal Salts with Mixed Anions

Summary.

This example demonstrates that metal salts composed of mixed anions of varying coordination strength can be utilized to increase the sensitivity and selectivity of adsorbate-induced anchoring transitions of liquid crystals (LCs) supported on surfaces decorated with the metal salts. Specifically, the dynamics of anchoring transitions triggered by the adsorbate dimethyl methylphosphonate (DMMP) are analyzed within the framework of a model for mass transport to demonstrate that the sensitivity of a nitrile-containing LC to DMMP increases from 250 ppb to 25 ppb on a surface of aluminum (III) cations, as the composition of the (counter) anion changes from 100% perchlorate to 90% nitrate and 10% perchlorate (by mole percent). Although the disclosed devices and methods are not limited by any specific theory or mechanism of action, additional experiments indicate that the anion of the metal salt changes the overall strength of coordination of the LC-aluminum (III), thus allowing for the LC in the coordination complex to be displaced by lower concentrations of DMMP.

Significantly, we also found that incorporating the nitrate anion into the metal salt decreases the influence of changes in humidity on the dynamic response of the LC anchoring transition to DMMP, a result that is consistent with weaker interactions between the nitrate anion and water when compared to interactions between the perchlorate anion and water. This interaction between the nitrate anion and water allows the metal cation to have stronger interactions with the LC or the adsorbate DMMP.

Finally, the bidentate anion acetylacetonate was found to cause a similar increase in sensitivity to DMMP when mixed with perchlorate in a 1:1 ratio (the resulting sensitivity of the system to DMMP was 100 ppb). These results demonstrate that tailoring the identity of the anions is an effective and facile approach to tuning the response of LCs supported on metal salts to targeted analytes.

Introduction.

Liquid crystals (LCs) and the interactions of the LC films with interfaces can be used as a detection scheme to detect analytes, including those in the vapor phase. Careful consideration must be given to the interactions between the surface and the LC that influence the response of the LC film to a targeted vapor analyte and to other interfering agents.

The alignment of thin films of nematic LCs supported on chemically functionalized surfaces is defined by intermolecular interactions between the surface and the LC. For example, coordination interactions between surfaces decorated with metal salts and nitrile-containing LCs (e.g., 4-cyano-4′-pentylbiphenyl, 5CB) result in a homeotropic alignment of the LC (parallel to surface normal) at the metal salt interface. The alignment of the LC is dependent on the strength of the coordination complex formed between the LC and metal salt, which is related to, among other factors, the electron affinity of the cation.

Specifically, the strength of the coordination complex is controlled by the combination of the strength of the individual interactions between the metal cation and LC and the number of interactions between the metal salt and LC. This proposition is supported by a previous study, which demonstrated that a homeotropic alignment of 5CB at the metal salt solid interface is obtained when the electron affinity of the cation of perchlorate salts exceeds a value of 15.6 eV. In the presence of targeted adsorbates in the vapor phase above the LC film, the coordination complex between the cation and LC is disrupted by the mass transport of the analyte from the vapor phase to the solid surface and the subsequent interaction of the analyte with the cation at the solid surface. This process occurs if the adsorbate coordinates more favorably with the metal salt in comparison to the LC-metal salt coordination complex. The disruption of the coordination complex triggers a change in the alignment of the LC at the solid surface to a planar alignment (perpendicular to surface normal).

For example, the analyte dimethyl methylphosphonate, DMMP, induces a change in the orientation of 5CB films in contact with aluminum (III) perchlorate salts decorated on solid surfaces from a homeotropic to a planar alignment. The vapor analyte DMMP is studied because it contains the phosphonate chemical moiety that is common in some nerve agents such as sarin. Detection of these types of analytes in the low parts-per-million (ppm) concentrations is important because the lethal concentration time for 50% of humans by inhalation for sarin is about 10-15 ppm min⁻¹.

The majority of previous studies have focused on high electron affinity cations, such as aluminum (III), nickel (II), and copper (II) that are paired with weakly coordinating anions, such as perchlorate, in order to obtain a homeotropic alignment of the LC film exposed to air. However, the choice of anion is important because the anion, in addition to the electron affinity of the metal cation, impacts the strength of the coordination complex that can form between the LC and metal cation, resulting in different orientations of the LC at the metal salt surface. The strength of the coordination complex formed between the metal salt and the LC is dependent on the number of interactions between metal salt and LC, along with the strength of the individual interactions between the metal salt and LC. For example, metal salts composed of a more strongly coordinating anion relative to the perchlorate anion, such as nitrate, chloride, or acetate, cause a planar orientation of LC within a supported, micrometer-thick film (approximately 90° from surface normal).

Conversely, other weakly coordinating anions including PF₆⁻ and BF₄⁻ have resulted in a homeotropic alignment of the LC when paired with a high electron affinity cation. We correlate the strength of anion coordination to the basicity constant of the anion, a parameter that measures the equilibrium between the anion and the hydrogen ion. A large basicity constant indicates equilibrium towards the complex with the hydrogen ion, which suggests strong coordination.

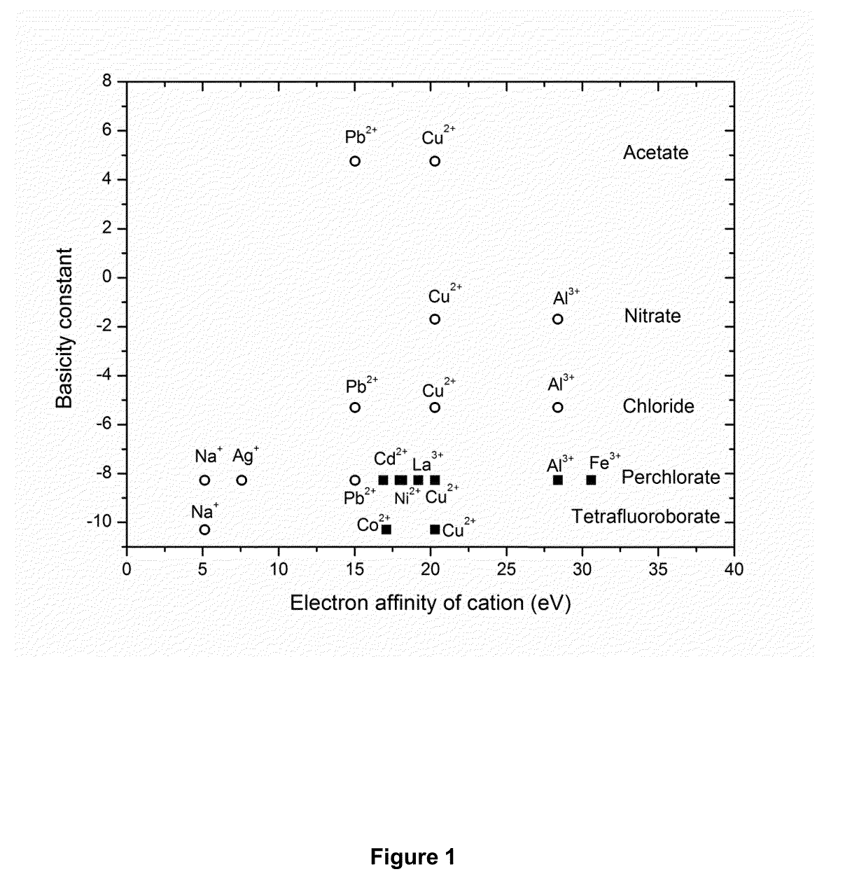

A summary of these key results of the alignment of nitrile containing LCs in contact with various metal salts from previous studies is illustrated in FIG. 1. The behavior between cations and anions is complicated by many factors involving the specific interactions between the electron acceptor and electron donor; however, the overall behavior of these interactions is summarized by the electron affinity of the cation and the basicity of the anion. In this example, we investigated the influence of the interaction between the cation and anion and their effects on the coordination of the cation with other ligands (i.e., nitrile group of LC and phosphoryl group of DMMP). Specifically in this example, we substantially expand the scope of the previous studies by exploring the effects of anion composition in metal salts with mixed anions on adsorbate-triggered anchoring transitions of LCs.

This example focuses on the role of Al³⁺ and Cu²⁺ salts comprised of various anions. These two cations were selected because a homeotropic alignment of the nematic 5CB has been previously reported on solid surfaces decorated with perchlorate salts of these cations. In addition, the coordination complex formed between the perchlorate salts of these cations and 5CB have been shown to be disrupted by the presence of DMMP. The strongly coordinating anions used in this example include nitrate, chloride, and acetylacetonate, all of which possess basicitiy values larger than the perchlorate anion. The anion acetylacetonate is a bidentate ligand that was chosen in order to determine any differences in the behavior of the anion based on changes in the number of binding sites.

In this example, we investigated the role of the anion by performing three different experiments. First, we characterized the orientations of LC films (of nematic 5CB) that are supported on aluminum (III) salts comprised of mixtures of both weakly coordinating anions and strongly coordinating anions. Second, we studied the response of LC films supported on metal salts of varying anion composition to the model analyte DMMP. DMMP is known to disrupt the metal salt-LC coordination complexes with weakly coordinating anions. Third, we measured the impact of a non-targeted analyte, water vapor, on the dynamics of the LC ordering transition triggered by DMMP. The results reported in this example suggest that the control of anion composition (using mixed anions) provides a facile method to tailor the properties of the coordination complex formed at the metal salt-decorated surface, which in turn improves the sensitivity and selectivity of the LC anchoring transitions to the targeted chemical analyte.

### Experimental Section

### Materials

11-Mercaptoundecanoic acid (MUA), aluminum (III) perchlorate nonahydrate, aluminum (III) nitrate nonahydrate, copper (II) perchlorate hexahydrate, and copper (II) chloride dehydrate salts were purchased from Sigma Aldrich (Milwaukee, Wis.). Aluminum acetylacetonate was purchased from Alfa-Aesar. 5CB was purchased from EMD Chemicals (Gibbstown, N.J.). Titanium (99.999%) and gold (99.999%) were purchased from Advanced Materials (Spring Valley, N.Y.). Methanol and Fischer's Finest glass slides were purchased from Fischer Scientific (Pittsburgh, Pa.). Absolute ethanol (anhydrous, 200 proof) was purchased from Pharmco-AAPER (Brookfield, Conn.). All chemicals and solvents were of analytical reagent grade and were used as received without any further purification. All deionized water used in the study possessed a resistivity of at least 18.2 MΩ cm.

Methods.

Cleaning of Glass Substrates:

Glass microscope slides were cleaned according to published procedures using acidic “piranha” solution [70:30 (% v/v) H₂SO₄:H₂O₂ (30%)] [19]. Briefly, the glass slides were immersed in an acidic piranha bath at 60-80° C. for at least 1 h, and then rinsed in running deionized water for 2-3 min. The slides were then immersed in basic piranha [70:30 (% v/v) KOH: H₂O₂] and heated to between 60 and 80° C. for at least 1 h. Finally, the slides were rinsed sequentially in deionized water, ethanol, and methanol, and then dried under a stream of nitrogen. The clean slides were stored in an oven at 110° C. All other glassware was rinsed with distilled water and ethanol and dried under a gaseous stream of nitrogen.

Deposition of Thin Layers of Gold:

Semi-transparent films of gold with thicknesses of 200 Å were deposited onto piranha-cleaned glass slides mounted on a fixed holder within an electron beam evaporator (VEC-3000-C manufactured by Tekvac Industries, Brentwood, N.Y.). The angle of incidence of the gold ranged from 0° to 15° on the slides (measured from surface normal). A layer of titanium (thickness 80 Å) was used to promote adhesion between the glass microscope slides and the films of gold. The rates of deposition of gold and titanium were 0.2 Å/s. The pressure in the evaporator was maintained at less than 3×10⁻⁶ Torr before and during each deposition. The gold source was periodically cleaned by sequential immersion in aqua regia (70% HNO₃, 30% HCl) and piranha solutions at 50° C. (30 min in each solution); see above for compositions. The cycle was repeated 3-4 times, rinsing the source between cycles in deionized water.

Preparation of Functionalized Gold Surfaces:

Carboxylic-acid-terminated self-assembled monolayers (SAMs) of 11-mercaptoundecanoic acid (MUA) were formed on gold-coated glass slides by immersing the slides overnight in an ethanolic solution containing 2 mM of MUA. The gold films were then rinsed with an excess of ethanol and dried under a stream of nitrogen. Mixtures of the metal salt were formed in an ethanolic solution in which the concentration of the metal cation remained constant at 5 mM. The percent of the anion reported is also in a ratio of mole percentage. The metal salts were then deposited immediately onto the carboxylic-acid-terminated SAMs by spin coating the 5 mM ethanolic solution of the salt solution at 3000 rpm for 60 s (WS-400A-6NPP/Lite, Laurell Technologies, North Wales, Pa.).

Formation of Micrometer-Thick Films of LC:

After coating the surfaces with metal salt, as described above, an 18 μm-thick gold-coated transmission electron microscopy (TEM) grid (Electron Microscopy Sciences, Hatfield, Pa.) was fastened to the salt-coated surface using a thin stainless steel plate (0.44 mm thickness). The stainless steel plate contained a 2-mm diameter hole that was aligned with the center of the TEM grid. The TEM grid defined square pores with lateral dimensions of 285 μm. The grid had an overall diameter of 3 mm. Both the TEM grid and stainless steel plate were dip-coated with a perfluorocarbon film (Nyebar Fluorocarbon Barrier Film, SmartGrease Company, Fairhaven, Mass.) to minimize wicking of the 5CB from the TEM grid. The grids were filled with LC using a microcapillary tube at room temperature, taking care to fill only the middle squares of the TEM grid, so as to avoid wicking of the 5CB between the TEM grid and steel plate.

Exposure to DMMP:

The sample, prepared as described above, was exposed to a stream of air containing DMMP within a flow cell that was constructed to direct the flow of air across the LC samples while permitting simultaneous observation of the samples through a polarized light microscope (CH40, Olympus, Melville, N.Y.). The stream of gas containing DMMP was generated using a certified cylinder containing 10 ppm DMMP in nitrogen (Matheson Tri-Gas Inc, Eagan, Minn.). That stream was diluted by air at a specified relative humidity, RH. The RH of the air was controlled using a portable dew point generator (LI-610, LI-COR Biosciences, Lincoln, Nebr.). The temperature of the gas fed to the flow cell was maintained at room temperature (25° C.) and the RH was controlled to 30%. The flow system was plumbed using 1/16″ stainless steel Swagelok tubing (Badger Fluid System Technologies Milwaukee, Wis.). The flow rate of the gas through the exposure system was controlled using a series of rotameters (Aalborg Instruments & Controls, Inc, Orangeburg, N.Y.). The volumetric flow rate of the 10 ppm DMMP stream from the cylinder was controlled between 0 to 150 mL/min, while the volumetric flow rate of the diluent air stream was controlled between 0 to 1000 mL/min. The concentration of DMMP used in the study reported in this paper ranged from 0.1 ppm to 1.25 ppm, while the overall volumetric flow rate of the gas stream passed over the samples remained constant at 1000 mL/min.

The time for the DMMP to travel from the gas cylinder to the flow cell was calculated from knowledge of the volumetric flow rate of the gas and the volume of the tubing in the flow system upstream of the sample. The length of the tubing running from the DMMP gas cylinder to the mixing point (where the stream was diluted with air, as needed) was approximately 120 cm, and after leaving the mixing point, the length of the tubing running to the flow cell was approximately 5 cm. When the volumetric flow rate of gas to the flow cell was 1000 mL/min, the time for DMMP to flow from the source cylinder (10 ppm) to the flow cell and mix with air to a concentration of 1 ppm was calculated to be 7 seconds.

The flow cell was fabricated by machining a rectangular prism of aluminum metal. Glass windows allowed transmission of polarized light through the flow cell. The intensity of light that was transmitted through the LC sample in the flow cell was quantified using an Olympus camera (Olympus C2040Zoom, Melville, N.Y.). The total volume of the flow cell was 10.5 cm³ (6 cm×3.5 cm×0.5 cm), giving rise to a residence time of ˜0.6 sec when the flow rate was 1000 mL/min. The Reynolds number was calculated to be 51 for airflow within the flow cell at 1000 mL/min. This value corresponds to laminar flow.

Birefringence Measurements of Sandwich Cells of 5CB:

The birefringence of thin films of 5CB was measured for the thin films in between metal salt decorated surfaces. These metal salt decorated surfaces were decorated with varying compositions of the metal anion. The sandwich cells were separated by an approximately 18 μm thick mylar spacer. The exact thickness of the sandwich cells was measured using an interferometry technique using visible light. The sandwich cells were then filled with 5CB and the retardance of the cells was measured using a compensator. From these values the birefringence of the samples is obtained and the value of the tilt angle of the 5CB using the known birefringence of 5CB.

Fourier Transform Polarization-Modulation IR Reflectance-Absorbance Spectroscopy (PM-IRRAS):

IR spectra of 8CB on surfaces were obtained using a Nicolet Magna-IR 860 FT-IR spectrometer with a photoelastic modulator (PEM-90, Hinds Instruments, Hillsboro, Oreg.), a synchronous sampling demodulator (SSD-100, GWC Technologies, Madison, Wis.), and a liquid-N₂-cooled mercury cadmium telluride (MCT) detector. All spectra were recorded at an incident angle of 83° with the modulation centered at 2200 cm⁻¹. All spectral data reported within this article were recorded within 500 cm⁻¹ of the modulation center. Data were collected in terms of differential reflectance vs. wave number, and spectra were normalized and converted to absorbance units via methods known in the art outlined in Frey et al. (“Polarization modulation approaches to reflection absorption spectroscopy.” Handbook of Vibrational Spectroscopy, J. Chalmers and P. R. Griffith; Wiley: New York, 2001).

Results.

1. Effect of Metal Salt Anions on the Alignment of 5CB Films in Air

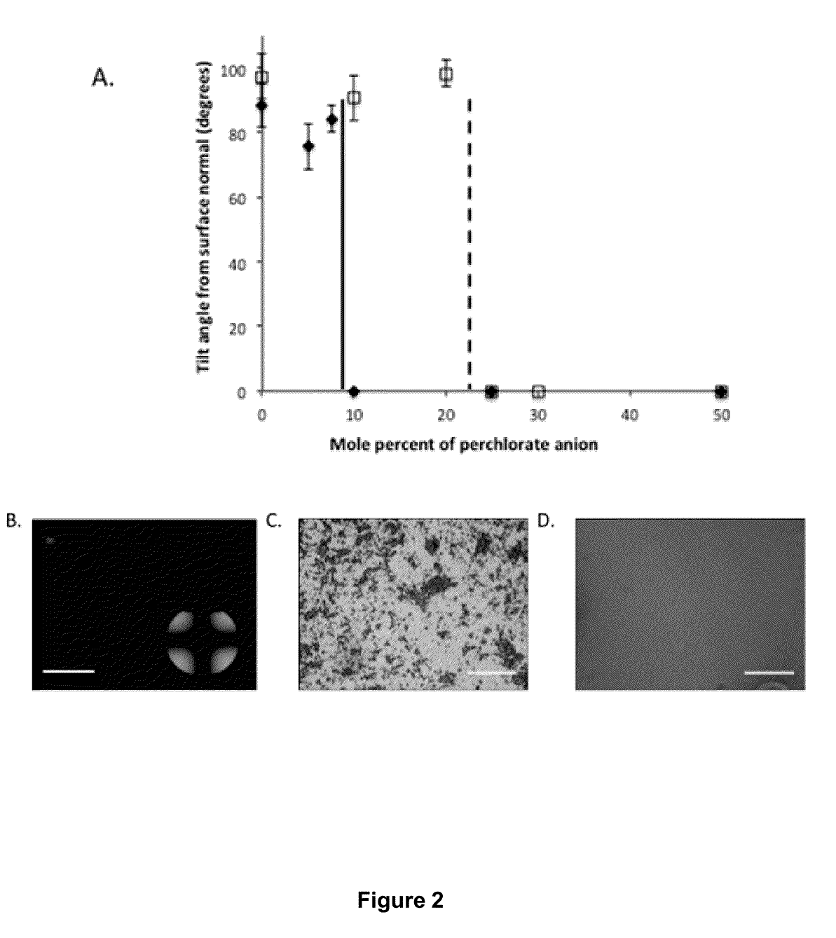

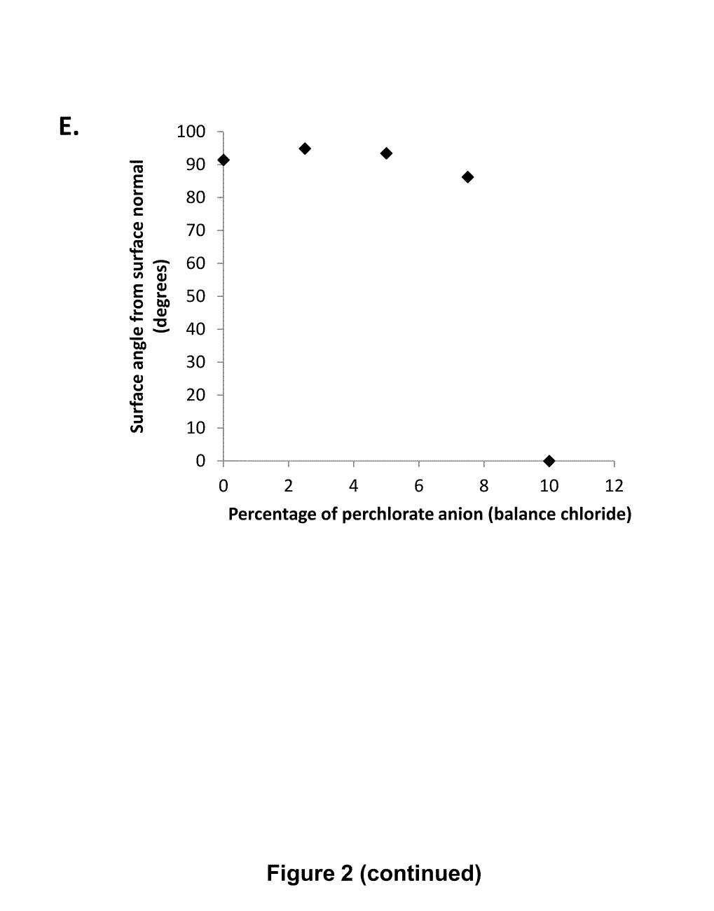

Our first set of measurements was designed to analyze the role of the anion of aluminum (III) salts on supported films of 5CB. Previous studies have identified the role of the electron affinity of the cation and basicity constant of the anion on the alignment of supported films of 5CB. Our first observation shown in FIG. 2 expands upon these previous results by investigating the anchoring of 5CB at the surface of aluminum (III) salts with mixed anions. For this example, we measured the birefringence of approximately 18 μm thick films of 5CB sandwiched between surfaces that are decorated with metal salt mixtures. From these measurements, we calculated the average tilt angle of 5CB at the metal salt surface as shown in FIG. 2A. The thickness of 18 μm for the LC films is estimated to be large enough to prevent deviations of the tilt angle at the metal salt surface.

The mixed anions were mixtures of a weakly coordinating anion (perchlorate) and a more strongly coordinating anion (either nitrate or acetylacetonate). FIG. 2A shows that the tilt angle of LC films at the aluminum (III) nitrate-coated surface is close to 90° from surface normal (within error), a result that is consistent with previous studies. As the mole fraction of perchlorate in the salt increased to just less than approximately 10%, the tilt angle of the LC at the interface did not vary greatly from 90°. We attribute these measurements of a tilt angle greater than 90° to an error in the measurement and system, and we associate the angle of 5CB at the metal salt surface to be close to planar. However, at a critical composition of the anions (around 10% perchlorate, 90% nitrate by mole percent), there is a discontinuous change in the alignment of the LC at the interface with the LC adopting a homeotropic alignment (tilt angle of zero). This critical composition is shown in FIG. 2A as the vertical solid black line.

Polarized light micrographs of the LC films in contact with surfaces composed of 100% perchlorate, 8% perchlorate/92% nitrate, and 100% nitrate are shown in FIG. 2B, C, D respectively. These optical micrographs are consistent with the behavior shown in FIG. 2A. FIG. 2B shows a dark image when viewed through cross polarizers for 5CB in contact with 100% perchlorate that is indicative of homeotropic alignment of the LC. This behavior is further confirmed by the cross-hair pattern when viewed through conoscopy shown in the inset of FIG. 2B. The image in FIG. 2C shows a polarized light micrograph of an LC film supported on films that contain 8% mole percent perchlorate and 92% nitrate anion that is indicative of the formation of domains within the sample. This is consistent with the idea that the transition from a homeotropic to a planar alignment of the LC is abrupt as the composition of the metal salt changes from a salt containing a weakly coordinating anion to a salt containing a mixture of the weakly coordinating anion and strongly coordinating anion. Therefore, we would expect to see the formation of domains in the sample instead of a gradual transition of the tilt angle of the LC from a planar to homeotropic alignment.

Furthermore, if the metal salt only contains a strongly coordinating anion such as the nitrate anion, a bright appearance of the LC is observed when the LC film is viewed through cross-polarizing films indicating a planar alignment of the 5CB. This result supports the proposition that the anion associated with the metal cation influences the strength of the coordination complex the cation has with other environmental species (i.e. the LC). In addition, a minimum strength of the coordination complex is required in order to obtain a homeotropic alignment of the LC at the metal salt surface. We have confirmed this behavior with additional studies of other metal salts, such as mixtures of Cu (II) perchlorate and Cu (II) chloride, as seen in FIG. 2E.

Next, we sought to characterize the mixed anion system by investigating mixtures of the weakly coordinating anion perchlorate and the more strongly coordinating anion acetylacetonate. We choose the anion acetylacetonate because it is a bidentate anion that can interact with the cation at two independent coordination sites. FIG. 2A shows a similar behavior of the acetylacetonate/perchlorate system when compared to the nitrate/perchlorate system. However, a key difference is the observation of a critical composition that was almost double the amount of perchlorate (around 25% perchlorate, 75% acetylacetonate by mole percent) as compared to the nitrate-perchlorate system. This general change in the critical composition of the perchlorate anion is consistent with the idea that each individual acetylacetonate molecule interacts with the aluminum (III) cation at two different coordination sites. Therefore, each individual acetylacetonate disrupts twice the number of coordination sites when compared to a unidentate strongly coordinating anion. Since each acetylacetonate molecule interacts with the cation at two independent binding sites, a greater amount of the perchlorate is required to give sufficient coordination interactions associated with the perchlorate anion to allow interactions between the metal salt and 5CB to form a coordination complex that results in a homeotropic alignment of 5CB.

The results in FIG. 2 suggest that the strength of the coordination complex formed between the metal salt and LC is dependent on the anion of the system. We relate the strength of the coordination complex between the metal salt and 5CB to the alignment of 5CB at the metal salt surface. The strength of the coordination complex is dependent on both the number of interactions between the metal salt and LC in addition to the strength of the individual interactions between the metal salt and LC. A homeotropic alignment of 5CB at the solid surface is a result of a strong coordination complex between the metal salt and the LC. In contrast, a weak coordination complex between the solid surface and LC results in a planar alignment of the LC.

Although the disclosed devices and methods are not limited by any theory or mechanism of action, two possible physical scenarios may explain the observations seen in FIG. 2. The first possible explanation is that changes in the anion of the salt influence the number of coordination interactions between the salt surface and the LC. The more strongly coordinating anion interacts strongly with the cation and prevents the cation from having coordination interactions with the LC. Therefore, fewer interactions exist between the metal salt and LC. There exist a critical number of interactions that are necessary to form a coordination complex between the metal salt and LC that is of enough strength to align the LC homeotropically as shown in FIG. 2. Alternatively, the impact of the strength of the coordination complex based on the introduction of a strongly coordinating anion could impact the strength of the individual interactions between the LC and the metal salt. Specifically, the more strongly coordinating anion decreases the strength of the individual interactions between the metal cation and LC. This decrease in the strength of the individual interactions does not have an effect on the number of interactions that are obtained at the surface. Therefore, the strength of interactions is dependent on the ratio of weakly coordinating to strongly coordinating anion of the metal salt. There exists a minimum strength of interactions between the metal salt and LC that is needed to obtain a sufficient coordination complex between the LC and metal salt in order to obtain a homeotropic alignment of the LC.

In order to distinguish between these two hypotheses, we characterized the interactions between the nitrile group and the metal salt with mixed anions using PM-IRRAS. To obtain these results, we made two necessary changes to the experimental system. First, the nitrile containing nematic LC 5CB was replaced by the nitrile containing smectic LC 4-cyano-4′octylbiphenyl (8CB) in order to obtain stable thin films of the LC around 1000 nm that were needed for the PM-IRRAS technique. Second, the Al (III) salts were exchanged for Cu (II) salts of mixed anions (perchlorate and chloride) in order to provide spectroscopic evidence of the coordination interactions between the copper cation and the nitrile vibration of the 8CB. Our data demonstrate similar that mixed copper (II) salts had a similar effect on the tilt angle as a function of the surface composition of anions as the mixtures of aluminum (III) salts (FIG. 2E).

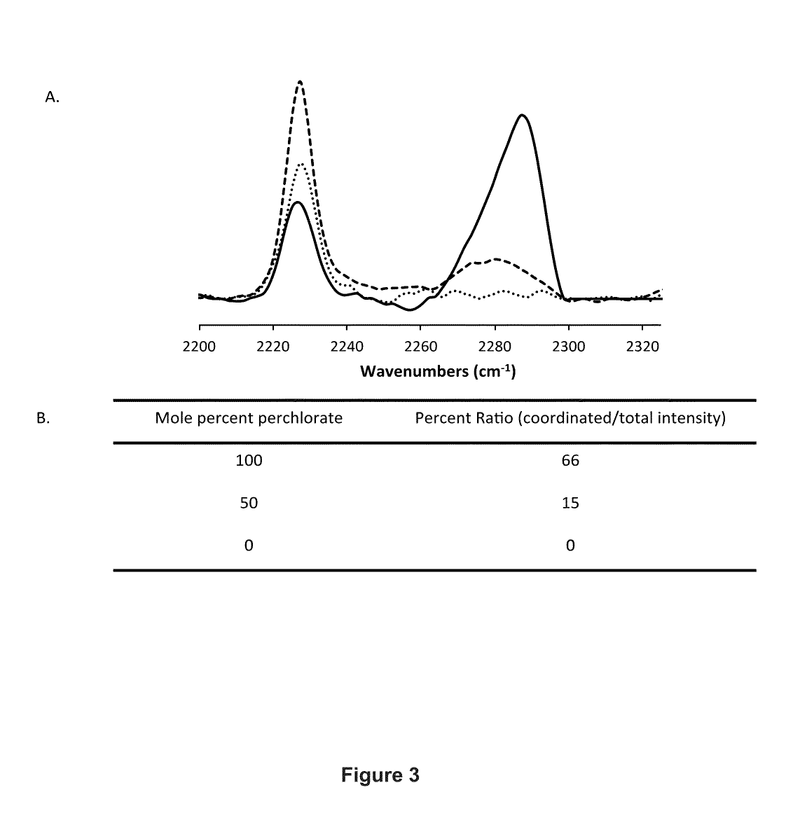

The spectra centered around the vibration of the nitrile chemical moiety of 8CB in contact with Cu (II) salts was studied in order to observe the intensity of the nitrile vibration frequencies in both the free and coordinated vibrational state. As FIG. 3 shows for 8CB in contact with 100% copper perchlorate, a free nitrile peak is observed at 2227 cm⁻¹ in addition to a coordinated nitrile peak at around 2287 cm⁻¹ that is related to the 8CB that is interacting with the copper (II) perchlorate salts. This result is consistent with previous studies. The ratio of the intensity of the coordinated nitrile peak to the total intensity of the both of the peaks was 66%. However, when the salt surface contains a 50/50% mixture of copper (II) perchlorate/chloride the intensity of the peak representing the coordination interaction between the LC and copper cation decreases while the free nitrile peak increases, indicating that there is a lower number of coordination interactions between the metal salt surface and the 8CB. This is indicated by the decrease to 15% of the ratio percentage of the intensity of the coordinated nitrile peak to the total intensity.

There is also a slight shift in the location of the peak to an intensity of 2279 cm⁻. The location of the free nitrile peak remains constant at 2227 cm⁻¹. This shift indicates that the strength of the coordination interactions has decreased slightly. This conclusion is based on a previous study showing that the location of the peak related to the nitrile vibration in a coordinated state shifts to larger wave number as the electron affinity of different metal perchlorate salts increases. This previous study suggests that a peak at larger wave numbers is indicative of a stronger coordination interaction. Furthermore, 8CB supported on a surface decorated with 100% copper (II) chloride results in a further decrease in the intensity of the coordination peak to a ratio of about 0%. Because the position of the coordinated nitrile peak is only shifted a little but there is a significant decrease in the intensity of the coordinated nitrile peak (as evidenced by the ratio of the coordinated nitrile peak to the total intensity shown in FIG. 3B), the IR analysis supports the hypothesis that the number of coordination interactions between the nitrile moiety and the metal salt decreases as the composition of the anion changes from perchlorate to nitrate. However, the disclosed methods and devices make use of weakly coordinating anions independent of whether they reduce the number or strength of individual coordination interactions.

In conclusion, in order to obtain a homeotropic alignment of the LC at the interface, the coordination complex needs to contain a minimum number of interactions or minimum strength of individual coordination interaction between the LC and metal cation. The number of interactions is influenced by the ratio of weakly and strongly coordinating anions.

2. Effect of Metal Salt Anions on the Alignment of 5CB Films in Low Concentrations of DMMP

In the previous section of this example, we reported results that demonstrate that salts with mixed anions are used to define the alignment of LCs at the metal salt surfaces. This section expands upon these observations by studying how the anion composition impacts the response of the aligned LC system to the vapor analyte DMMP. As noted in the Introduction, previous studies have shown that DMMP transported from the vapor phase to the metal salt interface, can displace the LC from the coordination complex with the salt. This causes a change in the alignment of the LC at the solid interface. In the experiments described below, we test the proposition that a lower number of coordination interactions between the metal salt, achieved by using mixed anions, will result in an alignment transition of the LC that is more easily disturbed by DMMP.

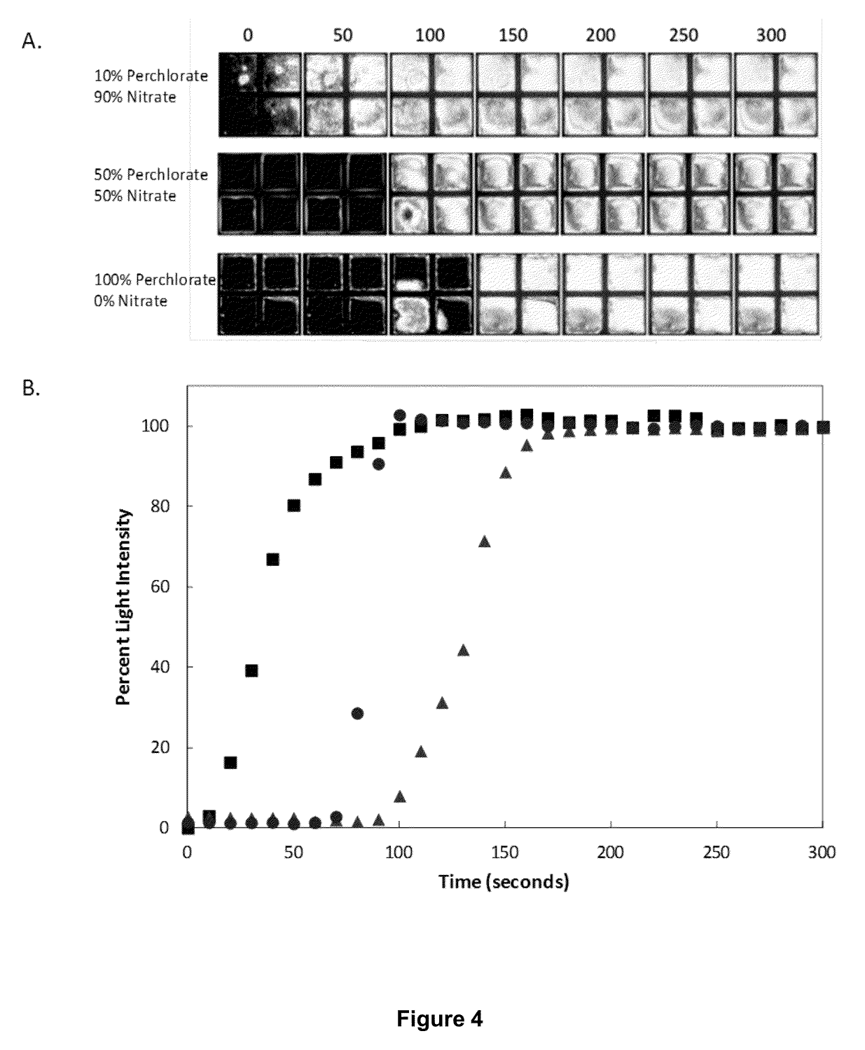

We tested this proposition by examining polarized light micrographs of thin films of 5CB supported on solid surfaces decorated with aluminum (III) salt of varying anion composition. The optical response of the supported LC film to exposure to DMMP was measured. FIG. 4A shows a series of polarized light micrographs of LC films in contact with aluminum (III) decorated surfaces, where the anion was 100% perchlorate, 50% perchlorate and 50% nitrate, or 10% perchlorate and 90% nitrate. Initially a dark appearance is observed in the optical micrograph that is consistent with a homeotropic alignment of the 5CB at the metal salt surface and a homeotropic alignment of the 5CB at the air interface. In the presence of DMMP, the optical micrographs appear bright indicative of a change in the alignment of the LC at the metal salt surface from homeotropic to a planar alignment. This transition is a result of the disruption in the coordination complex between the metal salt and LC by the presence of DMMP.

Inspection of FIG. 4A reveals that the time required to observe a visible change in the LC film increases as the amount of perchlorate anion involved in the system is increased. In order to quantify the changes in the time required to see a transition of the LC, four central squares of each LC film were analyzed and the average intensity of light transmitted through cross polarizers was measured, as shown in FIG. 4B (see Methods section). FIG. 4B clearly shows that the length of time required to observe a transition of the LC is influenced by the amount of the perchlorate anion in the system.

A previous study established that the LC coordination complex is not disturbed by DMMP until a critical concentration of DMMP, Cₘᵢₙ, is accumulated within the LC film. An estimate of Cₘᵢₙ can be obtained by measuring the time of response of the LC sensor system as the concentration of DMMP in the vapor phase is changed based on a simple mass transport model of the system. The final relationship between the time of response versus the concentration of DMMP in the vapor phase (C_{∞}) is described in Equation 1.

$${t}_{on , 0} = \frac{{δ}_{5 CB}}{{K}_{H} \cdot {k}_{ov}} ln ( \frac{{C}_{∞}}{{C}_{∞} - {C}_{min}} ) \qquad ( 1 )$$

This model requires knowledge of the thickness of the 5CB film, δ5CB, the partitioning coefficient DMMP from air to LC, KH, and an estimate of the overall mass transfer coefficient, kₒᵥ. In this study these parameters are estimated as the overall mass transfer coefficient as 0.24 cm/s, the partitioning coefficient of DMMP from the vapor to LC as 5.3×10⁻⁵, and the thickness of the 5CB film as approximately 18 μm.

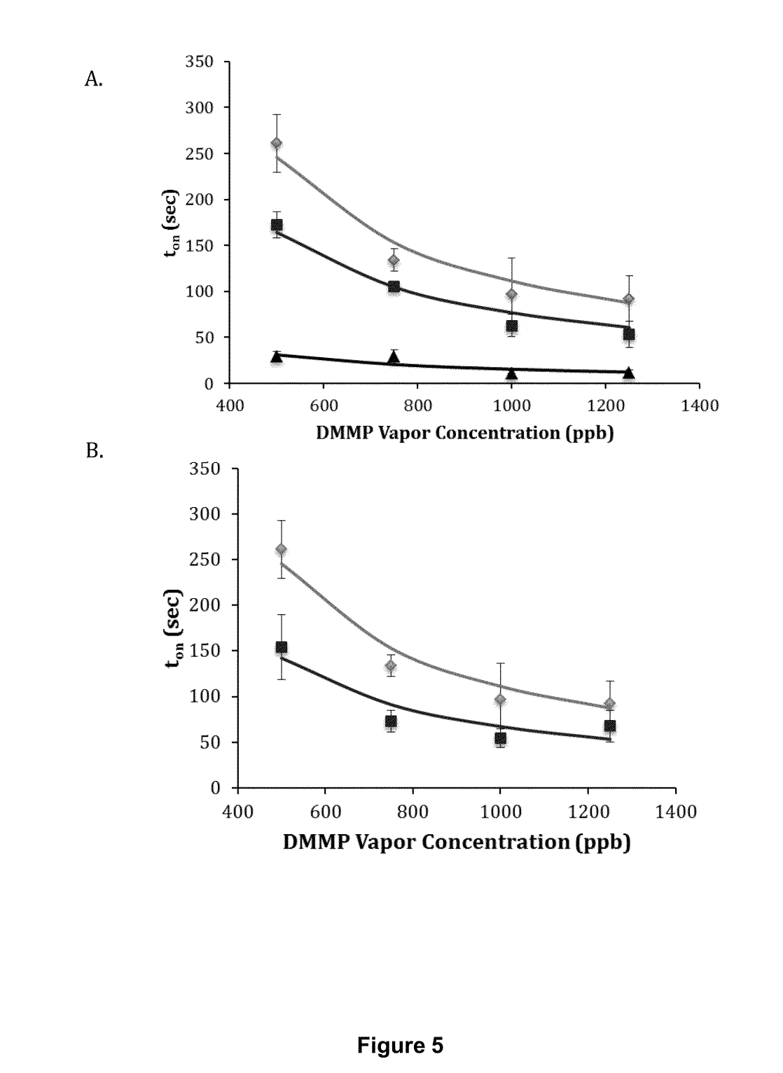

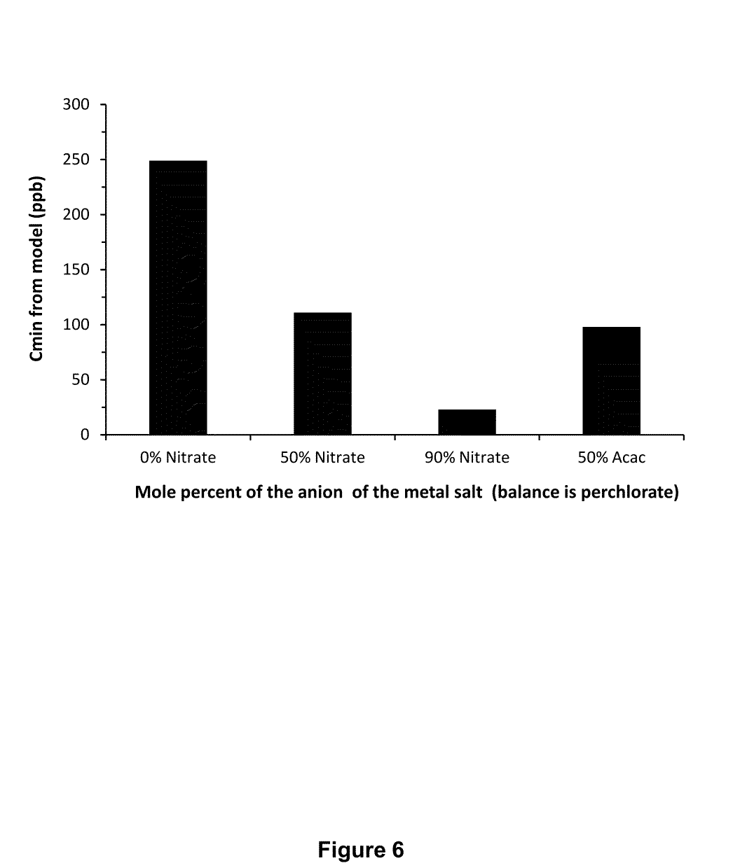

Based on the above-described model, the value of Cₘᵢₙ for the three different LC films supported on metal salt of mixed anions was estimated as a function of the composition of the anion by varying the concentration of DMMP in the vapor flowing over the LC (C_{∞}). The result is shown in FIG. 5A for three perchlorate/nitrate anion compositions. The decrease in the time of response as C_{∞} is increased for each of the individual systems is consistent with the behavior described in Equation 1 (solid lines). In addition, FIG. 5A reveals a significant decrease in the time of response of the LC as the composition of the anion contains an increase in the strongly coordinating anion within the metal salt. The values of the time of response were fit to the model proposed in Equation 1 and the value for Cₘᵢₙ was calculated for each of the vapor sensors. A 100% perchlorate surface result in sensitivity to DMMP that is calculated to be 250 ppb, whereas a 50% perchlorate/50% nitrate surface leads to a sensitivity of 110 ppb, and a 10% perchlorate/90% nitrate has the lowest sensitivity at 25 ppb as shown in FIG. 6.

To test these predictions, we measured the response of the LCs at the predicted sensitivities of the systems. The 100% perchlorate salt surfaces gave an optical response when exposed to a 300 ppb environment of DMMP, but no optical response was seen at lower concentrations such as at 200 ppb. This is in contrast to a 10% perchlorate/90% nitrate mixture, which resulted in an optical response of the LC when the sample was exposed to 100 ppb.

Similarly, FIG. 5B shows experimental results of a mixture of 50% acetylacetonate and 50% perchlorate (by mole percent). From this data, the sensitivity of the LC film supported by this metal salt anion composition to DMMP was calculated to be 100 ppb. This sensitivity is similar to that of a 50%/50% perchlorate/nitrate mixture suggesting that the influence of the number of binding sites does not influence the sensitivity of the salt surfaces to DMMP at similar compositions of the anion.

The results described above indicate that the sensitivity of the LC based sensor system increases as the composition of the more strongly coordinating anion increases relative to the perchlorate anion of the salt mixture that is supporting the film of 5CB. The values of the sensitivity are summarized in FIG. 6. In fact, as the composition of the metal salt approached the critical composition of the anion needed to obtain an initial homeotropic alignment of the LC, the sensitivity of the system increased by an order of magnitude of concentration of DMMP in the vapor phase (25 ppb for 90% nitrate, 10% perchlorate compared to 250 ppb for 100% perchlorate) for the nitrate/perchlorate mixed anion system.

The chemical reaction for the ligand exchange process that is occurring at the surface is given as

M-LC+DMMP→M-DMMP (2)

From the chemical reaction, we can write the equilibrium constant for the reaction that is occurring at the surface as:

$$K = \frac{[ M - DMMP ]}{[ M - LC ] [ DMMP ]} \qquad ( 3 )$$

In this case, the number of binding sites remains constant and therefore the addition of [M-DMMP] and [M-LC] must be constant. For the sample that is composed of the 100% perchlorate anion, we set the maximum number of binding sites to x_{max,100}. We set the critical number of [M-LC] to the value of xₘᵢₙ. If in the case of the 50% sensor, we have reduced the number of binding sites to be x_{max,50} and the threshold value of binding sites remains the same. If we assume that the equilibrium constant is equal for both of these cases, the equilibrium for both cases is evaluated as:

$$\frac{⌊ {x}_{max , 100} - {x}_{min} ⌋}{[ {x}_{min} ] [ {DMMP}_{100} ]} = \frac{⌊ {x}_{max , 50} - {x}_{min} ⌋}{[ {x}_{min} ] [ {DMMP}_{50} ]} \qquad ( 4 )$$

If we assume that the number of binding sites is reduced by one half for the 50% mole percent perchlorate sample then equation 4 is reduced to:

$$\frac{[ {x}_{max , 100} - {x}_{min} ]}{[ {DMMP}_{100} ]} = \frac{[ \frac{{x}_{max , 100}}{2} - {x}_{min} ]}{[ {DMMP}_{50} ]} \qquad ( 5 )$$

In order for equation 5 to be true, then [DMMP₁₀₀] must be greater than [DMMP₅₀] that is consistent with the threshold DMMP concentration measured and shown in FIG. 6. If we estimate the number of binding sites to the molar ratio of the perchlorate anion in comparison to the more strongly coordinating and set xmax,100 equal to 100 sites/(unit area) and x_{max,50} is equal to 50 sites/(unit area). For the perchlorate/nitrate system, xₘᵢₙ is equal to approximately 8 sites/(unit area). We then set the concentration of DMMP for the 100% sensor system to be the measured value of 250 ppb. If we set these values into equation 4, then we predict the sensitivity of the 50% perchlorate/50% nitrate system to give a sensitivity of 114 ppb and for the 10% perchlorate/90% nitrate to be 5 ppb. This trend is similar to the experimental measurements measured in FIG. 6. For the perchlorate/acetylacetonate, xmin increases to about 25 sites/(unit area). By substituting this value into equation 4, we calculate the sensitivity of the 50% perchlorate/50% acetylacetonate (based on the 100% perchlorate system) to be approximately 83 ppb. The experimental value measured is 100 ppb that is lower than that of the perchlorate/nitrate system that is similar to the model predictions.

The improved sensitivity of the LC sensor system comprised of solid surfaces decorated with mixtures of anions suggest that weaker anchoring based on a small number of interactions between the metal salt surface and LC are formed from using mixtures of weakly and strongly coordinating anions. This conclusion is consistent with the overall hypothesis that the interactions between the cation and anion influence the number of interactions of the cation with the other environmental species within the system such as the LC. Overall, this observation suggest that the sensitivity of the LC based sensor system can be increased by operating the sensor system using a coordination complex close to the minimum strength of the coordination complex required to obtain the homeotropic alignment of the LC in air by changing the number of interactions between the metal salt and LC.

3. Influence of the Metal Salt Anion on the Susceptibility of the Sensor System to Changes in the Relative Humidity of the Vapor Stream

Previous studies have shown evidence that the first coordination sphere of the nitrile moiety of acetonitrile with aluminum (III) perchlorate is disrupted by large concentrations of water. This suggests that the presence of other chemicals including water can interfere with the coordination complex between the LC and metal cation. However, a previous study using a salt-based LC sensor system showed that the sensor system was not disrupted by concentrations of water up to 75% R.H. We expand upon these results by investigating changes in the time of response as the humidity of the air is changed from 30% R.H. to 60% R.H. for the various salt mixtures used.

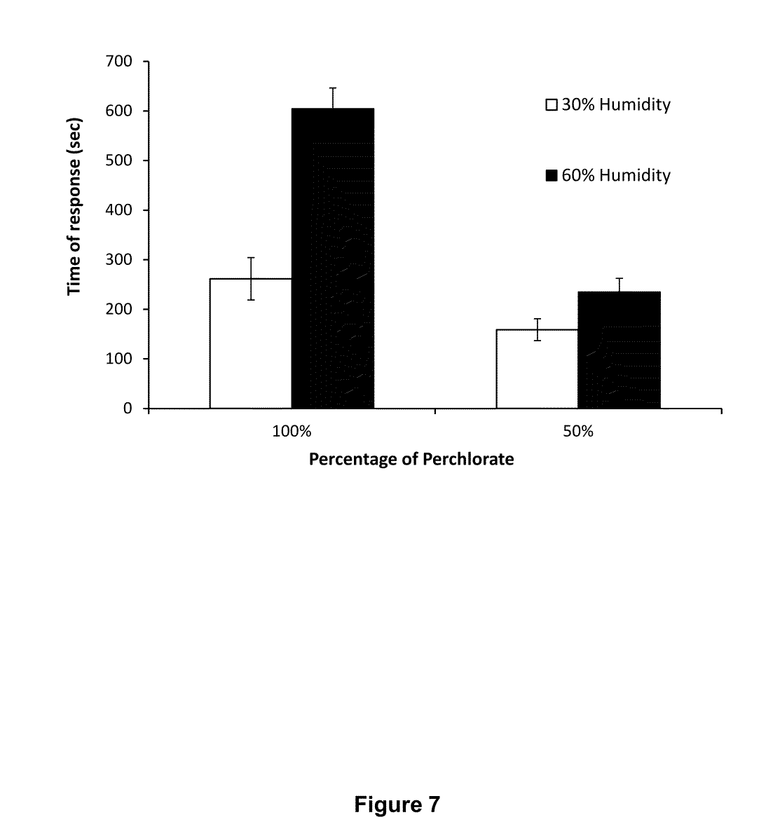

FIG. 7 shows the time of response for the 100% perchlorate salt system and a 50% perchlorate/50% nitrate salt system for both a 30% R.H. and 60% R.H. at a concentration of 500 ppb DMMP. For the case of the 100% perchlorate salt system, the time of response was measured to be 260 s at 30% RH, but increased to a value of 600 s at 60% RH (a change of 340 s or an increase of 230%). However, the 50%/50% perchlorate/nitrate mixture time of response was measured to be 160 s at 30% RH and increased to a value of 235 s at 60% RH (a change of 75 s or an increase of 150%). The change in time of response is lower for the 50%/50% nitrate/perchlorate when compared to the 100% perchlorate sample.

Although the disclosed methods and devices are not limited by any theory or proposed mechanism of action, we propose that this is due to the weaker interactions of water with the nitrate anion when compared to the interactions of water with the perchlorate anion. Water has weaker interactions with the nitrate anion because of the lower polarizability of the nitrate anion in comparison to the perchlorate anion. As the surface is exposed to the higher amount of water in the vapor, water will accumulate at the metal salt-LC interface. We hypothesize that this water will slow down the dynamics of the interactions of the metal salt with DMMP, thus causing a longer time required before an optical transition is observed. This will be a larger problem if the metal salt decorating the salt surface has strong interactions with water such as a 100% aluminum (III) perchlorate. However if the metal salt surface contains a metal salt that does not interact with water very strongly such as one containing the nitrate anion, then less water will be accumulated at the metal salt interface. In this case the DMMP will not be impeded from displacing the LC from interacting with the metal salt. Therefore, we would expect less of a change in the time of response for the higher humidities if the metal salt surface were decorated with a metal salt that has minimal interactions with water.

At higher concentrations of DMMP (1000 ppb), the change in time of response for both functionalized solid surfaces (100% perchlorate and 50%/50% perchlorate/nitrate) is minimal as the humidity increases from 30% RH to 60% RH. The difference for both samples at the higher concentration is around 40 seconds for both the 100% perchlorate and 50% perchlorate/50% nitrate sample. This indicates the perturbance of water is elevated at the lower concentration of DMMP.

The results presented in this section of the example show that incorporating anions that have weaker interactions with other species in the environment (such as water) results in a lower change in the time of response for the dynamics of the LC system at the lower concentrations of DMMP. These results suggest that interactions between the metal salt and other species such as water or other vapor analytes can impact the dynamics of the sensor to the targeted vapor analyte. Thus, optimal design of the solid surface of the LC sensor system requires careful consideration of the counter anion and the interaction of the anions with other potential environmental species, such as water.

The invention is not limited to the embodiments set forth in this disclosure for illustration, but includes everything that is within the scope of the claims. Furthermore, all documents cited in this disclosure are hereby incorporated by reference in their entirety and for all purposes as if fully set forth in this disclosure.

We claim:

1. A device for detecting a targeted agent, the device comprising:
a substrate;
one or more mixed metal salts disposed on the substrate, wherein the mixed metal salts each comprise one type of metal cation and two or more different types of anions; and
a liquid crystal in contact with the mixed metal salts.

2. The device of claim 1, wherein the two or more different types of anions include one or more strongly coordinating anions and one or more weakly coordinating anions.

3. The device of claim 2, wherein the one or more strongly coordinating anions are selected from the group consisting of CH₃COO⁻, C₅H₇O₂⁻ (acetylacetonate), Cl⁻, F⁻, Br⁻, SO₄²⁻, and NO₃⁻.

4. The device of claim 2, wherein the one or more weakly coordinating anions are selected from the group consisting of ClO₄⁻, BF₄⁻, PF₆⁻, SbF₆⁻, and B(OTeF₅)⁻.

5. The device of claim 1, wherein the metal cation is selected from the group consisting of Al³⁺, Ag¹⁺, Ba³⁺, Cd²⁺, Ce³⁺, Co²⁺, Cr³⁺, Cu²⁺, Eu³⁺, Fe²⁺, Fe³⁺, Ga³⁺, In³⁺, La³⁺, Mn²⁺, Ni²⁺, Pb²⁺, Pr³⁺, and Zn²⁺.

6. The device of claim 5, wherein the metal cation is Al³⁺ or Cu²⁺.

7. The device of claim 2, wherein the one or more strongly coordinating anions make up from about 5% to about 95% of the total moles of weakly coordinating anions and strongly coordinating anions present in the mixed metal salt.

8. The device of claim 7, wherein the one or more strongly coordinating anions makes up from about 50% to about 92% of the total moles of weakly coordinating anions and strongly coordinating anions present in the mixed metal salt.

9. The device of claim 8, wherein the strongly coordinating anion makes up from about 70% to about 91% of the total moles of weakly coordinating anions and strongly coordinating anions present in the mixed metal salt.

10. The device of claim 9, wherein the strongly coordinating anion makes up from about 75% to about 90% of the total moles of weakly coordinating anions and strongly coordinating anions present in the mixed metal salt.

11. The device of claim 1, wherein the metal cation is immobilized on the substrate.

12. The device of claim 1, wherein the liquid crystal is 5CB (4-n-pentyl-4′-cyanobiphenyl) or 8CB (4-cyano-4′octylbiphenyl).

13. The device of claim 1, further comprising a means for observing the orientational ordering of the liquid crystal.

14. A method for detecting a targeted agent in a sample, the method comprising:
(a) preparing a liquid crystal device according to claim 1;
(b) contacting the liquid crystal in the device with the sample; and
(c) observing the orientational ordering of the liquid crystal in the device;
wherein a change in the orientational ordering of the liquid crystal in the device indicates that the targeted agent is present in the sample.

15. The method of claim 14, wherein the two or more different types of anions included in the device comprise one or more strongly coordinating anions and one or more weakly coordinating anions.

16. A method for determining an optimal mole fraction of strongly coordinating anion in a mixed metal salt for use in a liquid crystal device for detecting a targeted agent, the method comprising:
(a) preparing two or more compositions comprising a mixed metal salt comprising one type of metal cation, a strongly coordinating anion, and a weakly coordinating anion, wherein each of the two more or more compositions includes a different proportion of strongly coordinating anion and weakly coordinating anion, as calculated by the mole fraction of the total moles of weakly coordinating anion and strongly coordinating anion present;
(b) disposing the two or more compositions onto separate substrates or onto separate regions of the same substrate;
(c) contacting the two or more compositions with a liquid crystal; and
(d) observing the orientational ordering of the liquid crystal in contact with each of the two or more compositions;
wherein the optimal mole fraction of the strongly coordinating anion is the mole fraction of strongly coordinating anion in the composition having the highest mole fraction of strongly coordinating anion that still exhibits a desired initial orientation.

17. The method of claim 16, wherein the desired initial orientation is a homeotropic liquid crystal alignment.

18. The method of claim 16, wherein the strongly coordinating anion is selected from the group consisting of CH₃COO⁻, C₅H₇O₂⁻ (acetylacetonate), Cl⁻, F⁻, Br⁻, SO₄²⁻, and NO₃⁻.

19. The method of claim 16, wherein the weakly coordinating anion is selected from the group consisting of ClO₄⁻, BF₄⁻, PF₆⁻, SbF₆⁻, and B(OTeF₅)⁻.

20. The method of claim 16, wherein the metal cation is selected from the group consisting of Al³⁺, Ag¹⁺, Ba³⁺, Cd²⁺, Ce³⁺, Co²⁺, Cr³⁺, Cu²⁺, Eu³⁺, Fe²⁺, Fe³⁺, Ga³⁺, In³⁺, La³⁺, Mn²⁺, Ni²⁺, Pb²⁺, Pr³⁺, and Zn²⁺.
